(19) United States
(12) Patent Application Publication
(10) Pub. No.: US 2022/0001076 A1
(43) Pub. Date: Jan. 6, 2022

(54) A DEGRADABLE COMPLEX OF SYTHETIC POLYMER AND NATURAL EXTRACELLULAR MATRIX FOR VASCULAR GRAFTS WITH RELATED PREPARATION METHODS

(71) Applicant: Nankai University, Tianjin (CN)

(72) Inventors: Deling Kong, Tianjin (CN); Xianhao Dong, Tianjin (CN); Meifeng Zhu, Tianjin (CN)

(73) Assignee: Nankai University, Tianjin (CN)

(21) Appl. No.: 17/256,198

(22) PCT Filed: Jun. 1, 2019
(86) PCT No.: PCT/CN2019/089734

§ 371 Date: Dec. 26, 2020

(30) Foreign Application Priority Data
Mar. 26, 2019 (CN) 201910230829.3

(51) Int. Cl. A61L 27/18; A61L 27/36; A61L 27/58; A61L 27/56; A61L 27/50

(52) U.S. Cl. CPC A61L 27/18; A61L 27/3633; A61L 27/507; A61L 27/56; A61L 27/58

(57) ABSTRACT

The invention relates to the complex of synthetic polymer and natural extracellular matrix for vascular grafts and their preparation methods. The components of biodegradable synthetic polymer in the preparation process can be chosen with different material proportions. The scaffolds with different fiber diameters, different fiber arrangements, different pore sizes and different pore structures can be prepared by electro-spinning, wet-spinning, melt-spinning, 3D printing, pouring, phase separation, particle leaching and other technologies. Among them, the natural extracellular matrix components come from a wide range of sources such as vascular tissues from different kinds of animals including arteries and veins of pigs and cattle or vascular tissues from human donors including umbilical cord vessels, etc. And its composition and content can be flexibly adjusted according to the demand. The composites and artificial vessels prepared by this technology not only have good mechanical properties, controllable spatial structure and suitable degradation rate, but also have excellent biocompatibility and bioactivity. The preparation process of the invention is simple, the controllability is high, the preparation condition is mild, and is suitable for large-scale industrial production.

### REFERENCE TO RELATED APPLICATION

This application claims the benefit of the filing date of PCT/CN2019/089734 filed on Jun. 1, 2019, entitled “A DEGRADABLE COMPLEX OF SYNTHETIC POLYMER AND NATURAL EXTRACELLULAR MATRIX FOR VASCULAR GRAFTS WITH RELATED PREPARATION METHODS”. The teachings of the entire referenced application is incorporated herein by reference.

### FIELD OF THE INVENTION

This invention belongs to the field of tissue engineering. Specifically, it involves the complex of synthetic polymer and natural extracellular matrix for vascular grafts and their preparation methods

### BACKGROUND OF THE INVENTION

Vascular disease is the leading cause of global mortality. Vascular diseases arise from reduced blood flow and lack of nutrients due to stenosis or obstruction of vasculature. This can result in tissue or organ damage. Vascular diseases include, but are not limited to, coronary heart diseases, cerebrovascular diseases, peripheral artery diseases and deep vein thrombosis. According to the World Health Organization, the number of people dying of cardiovascular related diseases is estimated to increase to 23.3 million every year by 2030. Vascular transplantation is the routine treatment for treating vascular diseases. The gold standard for this kind of operation is to use autologous vascular grafts, collected from the patient's own vasculature, from locations such as the saphenous vein, bilateral internal thoracic artery and radial artery. However, some patients require the use of small diameter vascular grafts, as autologous vasculature has already been used or health complications have compromised the vascular tissue. In addition, small diameter vascular grafts can also be used for hemodialysis to construct an arteriovenous fistula and used to repair damaged vasculature following traumatic arterial injury or peripheral aneurysms.

At present, the long-term patency rates of large-diameter (inner diameter>6 mm) vascular grafts prepared by Dacron, Gore-Tex and polyurethane materials are high, and these grafts have been widely used in clinical practice. However, the patency rate of small diameter vessels (inner diameter<6 mm) made from similar kinds of non-degradable materials remains too low for clinical application. Despite efforts by researchers to improve anti-coagulant performance of small diameter vascular grafts through modification (e.g. the use of heparin, the long term patency rates of these vascular grafts have yet to be improved. Consequently, the invention of new biodegradable small diameter vascular grafts has increasingly gained more attention from research teams across the world.

A series of chemically synthesized biodegradable polymer materials have previously been used to prepare small diameter vascular grafts, such as polycaprolactone (PCL), poly(L-caprolactone) (PLCL), polyurethane (PU), poly(glycerol sebacate) (PGS), poly(lactic acid) (PLA), poly(glycolic acid) (PGA), poly(lactic-co-glycolic acid) (PLGA), poly(p-dioxanone) (PDS), and poly(ethylene glycol) (PEG), among others. Compared with non-degradable materials, implanted biodegradable synthetic materials have the advantage of utilizing the host's tissue remodeling potential to generate quasi-natural vascular grafts in situ. This has made the use of biodegradable polymer materials an attractive vision for furthering the development of small diameter vascular grafts.

As the research field progresses, it has become clear that there still exists a series of problems with small diameter vascular grafts made of synthetic biodegradable polymer materials. The overall biocompatibility is not ideal and the biological activity is poor. The inductions of acute inflammatory reactions after implantation in vivo are not conducive to the adhesion, migration and proliferation of peripheral vascular cells, and is not conducive to material-tissue integration with natural vascular tissue. Thus, with the sole use of synthetic biodegradable polymer materials for small diameter vascular grafts, it still remains difficult to achieve natural regeneration of vascular tissues within a short timeframe.

In recent years, acellular extracellular matrix (ECM), derived from various tissues, has also been used as scaffold materials for tissue engineering repair. Allogeneic or xenogeneic skin, pericardial tissue, submucosal tissue of the small intestine, peritoneum or other collagen-based matrices have been used as acellular ECM scaffolds. Through the use of physical agitation, chemical surfactant treatment and enzymatic digestion; proteins, lipids and nucleotide residues are removed to effectively reduce the immunogenicity of the ECM scaffold. ECM scaffolds contain collagen, glycosaminoglycan, structural protein, bioactive growth factors, tissue-specific exosomes and other substances, which contribute to the creation of a beneficial cell niche within the damaged tissue site, thus promoting the adhesion, proliferation and differentiation of surrounding tissue cells.

Despite their advantages, ECM scaffolds generated from natural tissues often have relatively dense matrices, and their porosity and pore size are difficult to control. Thus, they are not conducive to the migration of vascular cells into the interior of the ECM scaffold. Consequently, it is difficult to achieve good integration with surrounding tissues. Furthermore, ECM materials have weak mechanical properties, making them unsuitable for use as scaffolds in certain tissues. When placed under internal loading mechanics of the tissue microenvironment, ECM scaffolds can disintegrate rapidly and the tissue will lose functionality. For vascular grafts, mechanical strain and tissue function are important, the loss of these together with difficulty of operation and suture, can easily lead to the occurrence of an aneurysm. Although the mechanical indices could be improved by chemical crosslinking, the problems of fracture, cytotoxicity and non-degradability in the later stage of implantation are remaining issues and can aggravate the degree of calcification observed in vascular grafts. In addition, the poor solubility of ECM in organic solvents can lead to difficulty in chemical or physical modification.

To address the above problems, artificial graft materials with good biocompatibility, tissue-appropriate mechanical strength and controllable porosity and pore size are required to realize the rapid regeneration of vascular tissue following small diameter vascular graft implantation.

### SUMMARY OF THE INVENTION

This invention involves the complex of synthetic polymers with natural extracellular matrix to form vascular grafts, and their preparation methods. During preparation, there are one or more ratios for the components of degradable synthetic polymers. Various scaffolds of different diameters, fiber arrangements, apertures and pore structures can be generated by using multiple fabrication technologies such as electrostatic spinning, wet spinning, melt spinning, 3D printing, pouring, phase separation, particle leaching, etc. Natural extracellular matrix has extensive sources; vascular tissue from different animal species (such as the arteries and veins of pigs and cattle) or from human donors (such as umbilical cord). In addition, the methods described here have flexibly, thereby adjustment of components and contents can meet the necessary demands. The complex of the materials and the vascular grafts obtained thereafter, have good mechanical properties, controllable spatial structure, suitable degradation rates, and excellent biocompatibility and biological induction ability. The simple preparation of this invention is controllable and utilized mild conditions; suitable for large-scale industrial production.

### BRIEF DESCRIPTION OF THE DRAWINGS

FIG. 1. Comparison of the morphology of different materials. (a) The bright field diagram; (b) The bottom view of the prepared ECM powder under scanning electron microscope (SEM); (c) The bottom view of the highly oriented single component PLCL micron fiber without ECM powder under scanning electron microscope (SEM); (d) The bottom view of the highly oriented single component PLCL micron fiber with ECM powder under scanning electron microscope (SEM).

FIG. 2 Fourier transform infrared spectroscopy of ECM powder and PLCL composite material containing various concentrations of ECM.

FIG. 3 Comparison of the prepared membrane scaffold which is made of pure PLCL material (left) and PLCL composite material containing ECM (right) after subcutaneous implantation in rats for one week.

FIG. 4 Stereoscopic microscope images after four weeks' transplantation of abdominal aorta in rats with (a) single component PLCL vascular grafts and (b) PLCL composite vascular grafts containing ECM.

FIG. 5 Staining images comparison after four weeks' transplantation of abdominal aorta in rats with single component PLCL vascular grafts (a, c) and PLCL vascular grafts containing ECM powder (b, d).

### DETAILED DESCRIPTION OF THE PREFERRED EMBODIMENTS

The technology problem solved by this invention is the provision of biodegradable synthetic polymer and natural extracellular matrix composites, vascular grafts, and their preparation methods. The synthetic polymers mentioned in this invention can be used in one or more material ratios, and scaffold materials with different fiber diameter, fiber arrangement, pore size and pore structure can be achieved by using electrospinning, wet spinning, melt spinning, 3D printing, phase separation, particle leaching and other technologies. Vascular grafts made in this was provide good mechanical properties, controllable spatial structure and suitable degradation rates for facilitating tissue regeneration. Therefore, problems such as weak mechanical properties, dense structure and instability of pure ECM materials used as vascular grafts, are solved. The natural ECM components mentioned could be selected from vascular tissues of different animal sources (such as arteries and veins of pigs and cattle) or vascular tissues of human donors (such as umbilical cord, etc.). They have a wide range of sources and can be flexibly adjusted to alter the composition and content of ECM, according to the tissues' demands. As ECM contains a large number of glycosaminoglycan, collagen, exosomes (which contain a variety of miRNA related to tissue regeneration and development) and other natural active components, it provides a level of biological activity, superior to inert synthetic degradable polymer materials. Thus, regulation of the inflammatory reactions (such as the M2 polarization of macrophages) and promotion of the proliferation and maturation of tissue cells can be achieved. In summary, the preparation technology not only has the advantages of facile processing steps and good mechanical properties from synthetic polymer materials, but also preserves the characteristics of biological induction activity of ECM-derived materials.

The invention discloses a composite material of degradable synthetic polymer and natural ECM, including 1 portion of ECM and 0.1-10 portions of synthetic polymer compound by mass fraction.

Furthermore, the synthesized polymer compounds include PCL, PLCL, PU, PGS, PDS, PGA, PLGA, PEG and PHA (poly hydroxy fatty acid ester), in any proportion.

Furthermore, the invention also discloses one kind of vascular grafts, which is prepared by using the degradable synthetic polymers and the natural extracellular matrix composite materials.

Furthermore, the invention also discloses a production method of the vascular grafts, which comprises the following steps:

Step 1, configuration. Mix the formula quantity of extracellular matrixes with the solvent and disperse evenly. Then add the formula amount of synthetic polymer compounds, and disperse evenly to make the mixed solution.
Step 2: stereotyping. The mixture is stereotyped by the stereotyping methods to prepare the vascular grafts.

Furthermore, the solvent adopts at least one or more arbitrary proportion mixtures of tetrahydrofuran, dichloromethane, trichlormethane, acetic acid, acetone, trifluoroethanol, hexafluoroisopropanol, N, N-dimethylformamide, etc.

Furthermore, the concentration of the extracellular matrix mentioned in the step 1 is 0.001-1.0 g/ml (extracellular matrix mass/solvent volume).

Furthermore, the stereotyping methods used are electrospinning, wet spinning, pouring, melt spinning, 3D printing, phase separation, particle leaching, etc.

Furthermore, the diameter of vascular grafts mentioned is 0.5-20 mm.

Preferably, when the setting method adopts melt spinning or 3D printing, step 2 mentioned adopts following method. Remove the mixed liquid mentioned in step 1 to get complex polymer materials with evenly separated ECM powder. Add these materials to constant temperature heating barrel, increase the temperature to melt the complex materials and regulate a series of parameters such as the three-dimensional (x, y, z-axis) moving track of the barrel, the piston speed of the barrel, the thickness of the needle, the rotating speed of the receiver and the transverse moving speed of the barrel to control the micron fiber diameter, and the angle between the fibers to get oriented fiber tubular scaffolds with diameter of 10-50 μM.

Preferably, when the stereotyping method adopts phase separation, step 2 mentioned adopts the following pattern. Pour the mixture mentioned in step 1 into a special mold, control the temperature and cool to separate the phases of the mixture, separate the mixed liquid mentioned, quench the bicontinuous polymer phase and solvent phase to form two-phase solid, and then remove the solvent in the solid phase by sublimation and/or solvent replacement, obtain porous tubular scaffolds by controlling the quenching time and phase separation mechanism.

Preferably, when the stereotyping method adopts particle leaching method, step 2 mentioned carries on as the following pattern. Disperse the required pore forming agent (insoluble in the mixed solution) into the mixture described in step 1 evenly, and adjust the amount and size of the porogen to regulate the porosity and pore size. Then pour the liquid into a special mode. After the solvent volatilized, the residual solvent in the mixture was removed by vacuum and/or freeze-drying method to obtain the dried polymer composites with evenly separated ECM powder and porogen. The porous tubular scaffolds could be obtained by vacuum drying after the porogen in the composite material being leached out with a leaching solvent (insoluble polymer).

Furthermore, the porogen could adopt at least one of sodium chloride, polyethylene glycol (PEO), maltose and glucose.

Furthermore, the leaching solvent adopts at least one of water, gradient ethanol.

The beneficial effects of this invention are as follows.

1. Compared with the pure synthetic polymer materials, this composite material contains natural active components, such as glycosaminoglycan, collagen and exocrine due to the addition of vascular specific extracellular matrix powder. It significantly improves the biocompatibility and bioactivity of the bioinert synthetic polymer materials and contributes to the rapid and good regeneration of vascular grafts after implantation.

2. Compared with pure ECM materials and because of the addition of synthetic polymer materials, the main mechanical indices such as tensile strength, elongation at break, suture strength and Young's modulus of this composite material are significantly improved. It fully meets the mechanical requirements of vascular grafts. At the same time, the degradation rates of materials can be controlled, which avoids rapid degradation of natural ECM materials in vivo, so that the degradation rates of the materials could be matched with the rate of tissue regeneration. In addition, the processability of the material is obviously improved, and a variety of scaffolds with different structures could be obtained, which solves a series of problems, including the relatively dense natural extracellular matrix, uncontrollable porosity and pore diameter, and inefficient migration of host cells to the interior of the material.

3. This preparation technology is highly controllable, and the vascular grafts with required structure and biochemical properties could be obtained by a variety of processing methods. It is suitable for the preparation of vascular grafts with different sizes and morphologies.

The technical protocol of the invention will be described clearly and completely in combination with the drawings below. it is obvious that the described embodiments are part of the embodiments of the invention rather than all embodiments of the invention. Based on the embodiments of the invention, all other embodiments obtained by ordinary technicians in the field without creative work fall within the scope of the protection of the invention.

The Raw Materials Used in this Invention Come from the Following Sources:
Extracellular matrix (ECM): ECM is obtained from different kinds of animal vascular tissue (such as arteries, veins of pig, bovine, etc.) or human donor vascular tissue (such as umbilical cord, etc.) in slaughterhouses or hospitals. It is obtained after decellularization.
Poly (l-lactide-caprolactone) (PLCL): viscosity: 2.6-2.8, ratio: 50/50, Jinan Daigang Biological Engineering Co., Ltd. (Jinan, Shandong, China).
Polycaprolactone (PCL): Molecular weight: 80,000, Sigma aldrich (St. Louis, Mo., USA).
Polylactic acid (PLA): Molecular weight: 40,000, Sigma aldrich (St. Louis, Mo., USA).
Polyglycerol Sebacate (PGS): laboratory synthesis.
Polyurethane (PU): Sigma aldrich (St. Louis, Mo., USA).
Polyethylene glycol (PEO): Molecular weight: 8,000; Sigma aldrich (St. Louis, Mo., USA).
hexafluoroisopropanol: 99+%, Alfa Aesar (London, England).
N, N-dimethylformamide: 99.9%, Alfa Aesar (London, England).
Chloroform: 99%, Tianjin Chemical Reagent Factory (Tianjin, China).
Methanol: 99.9%, Shanghai Aladdin Biotechnology Co., Ltd. (Shanghai, China).
Tetrahydrofuran: 99.9%, Shanghai Aladdin Biotechnology Co., Ltd. (Shanghai, China).
NaCl: 99.9%, Sigma aldrich (St. Louis, Mo., USA).

The Main Instruments Used by this Invention are as Follows:
Freeze dryer (Beijing Boyikang, China).
Freezing grinder (Shanghai Jingxin, China).
Homogenizer (Bertin Technologies, USA).
Analytical balance (Sartorious PB-10, Germany).
Magnetic agitator (Gongyi Yingyu Yuhua instrument Factory, China).
Microinjection pump (Cole Parmer, USA).
High voltage electrostatic generator (Tianjin Dongwen Power Plant, DW-P503-1 AC, China).
Wet spinning instrument (laboratory self-made).
Melt spinning instrument (laboratory self-made).
3D Printer (GESIM, Germany).
Circulating water multi-purpose vacuum pump (Zhengzhou Great Wall Science, Industry and Trade Co., Ltd., China).

The Detection Equipment Used by this Invention is as Follows:
Scanning electron microscope (SEM, Quanta200, Czech).
Fourier transform infrared spectroscopy (TENSOR II, Bruker, Germany).
Frozen slicer (Leica CM1520, Germany).
Optical inversion microscope (Leica DM3000, Germany).
Advanced positive position microscope (Zeiss Axio Imager Z1, Germany).
   The present invention is explained in greater detail in the following non-limiting examples.

### Example 1: Preparation of Poly (1-Lactide-Caprolactone) (PLCL) and Extracellular Matrix (ECM) Composite Bilayer (Oriented Inner Layer and Random Outer Layer) Vascular Grafts

Preparation of vascular grafts inner layer. 1.0 g ECM powder was added to 10 ml hexafluoroisopropanol, and the ECM powder was further homogenized by using a homogenizer, then 2.0 g PLCL was added to the solution, stirring and dissolving overnight at room temperature, then a mixed solution with a concentration of 20% (mass/volume) PLCL and 10% (mass/volume) ECM was obtained. The vascular grafts were prepared in the fume cupboard by wet spinning at room temperature. The stainless-steel receiver with 2.0 mm diameter was installed on the wet spinning instrument. The mixed solution was inhaled into the syringe. The syringe was installed on the injection pump, and the syringe needle was placed at the position of 5 cm from the receiver in the spinning coagulation bath. Set the speed of the syringe pump to 15 ml/h, the speed of the receiver to 3000 rpm, the moving speed to 1 mm/sec, and the spinning time to 20 min. After completion, take the inner layer off from the wet spinning instrument and place it into a vacuum dryer to remove the coagulation bath and spinning solution solvent.

Preparation of vascular grafts outer layer. 0.5 g ECM powder was added to 10 ml hexafluoroisopropanol, and the ECM powder was further homogenized by using a homogenizer. Then 1.0 g PLCL was added to the solution, stirring and dissolving overnight at room temperature to obtain a mixed solution with a concentration of 10% (mass/volume) PLCL and 5% (mass/volume) ECM. The outer layer of vascular grafts was prepared in the fume cupboard by electrospinning at room temperature. Specifically, the receiver with inner layer of vascular grafts was installed on the electrospinning instrument and grounded, the mixed solution was inhaled into the syringe, the syringe was installed on the injection pump, the syringe needle was placed in the position away from the receiver 20 cm, and the 7 kV voltage was applied on the needle using high voltage DC power supply. The propulsion speed of the injection pump was 10 ml/h. The rotational speed of the receiver was 500 rpm, and the spinning time was 10 min. After the preparation, the injection pump was removed from the electrospinning instrument and placed in a vacuum dryer to remove the solvent. After the completion, the tubes were removed from the receiver to form a double-layer vascular grafts product.

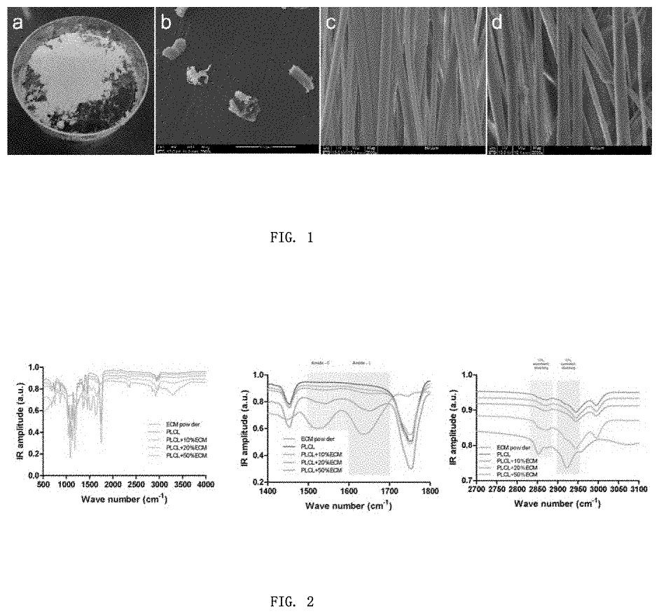

As shown in FIG. 1-5, the product of example 1 is tested.

FIG. 1 proves that the morphology of the composite produced by this method is similar to that of traditional materials.

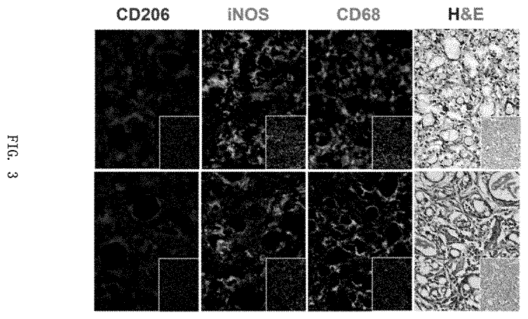

FIG. 2 proves that the composite material produced by this method could form a chemical bond between ECM and PLCL effectively.

FIG. 3 shows the analysis results of the scaffolds subcutaneous implanted in rats for a week, left panel is a single component PLCL fiber membrane scaffold, and the right panel is a PLCL fiber membrane scaffold containing ECM powder. The results of hematoxylin-eosin (H&E) staining and CD68 immunofluorescence staining show that the addition of ECM reduces the infiltration of inflammatory cells, increases the proportion of macrophage M2/M1, and significantly improves the biocompatibility of the scaffold.

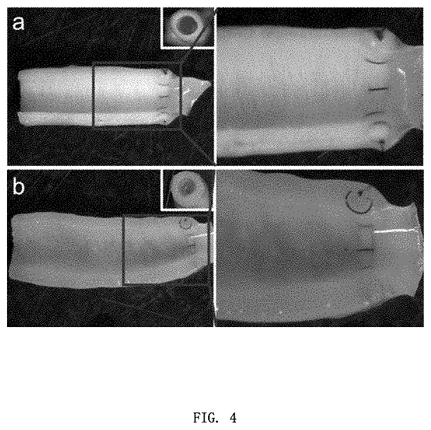

FIG. 4 shows the stereoscopic microscope images of the vascular grafts grafted into rats abdominal aorta for four weeks. Single component PLCL vascular grafts (a) still shows a white non-transparent material and clearly visible fibers which similar as it before implantation. The PLCL vascular grafts containing ECM powder (b) shows white and transparent tissue, and the vascular shows good remodeling.

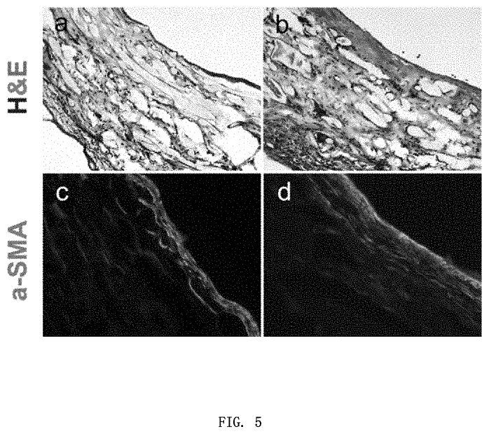

FIG. 5 shows the staining results of four weeks after the transplantation in rats abdominal aorta. Hematoxylin eosin (H&E) staining and a-SMA immunofluorescence staining (a, c) show poorer neointimal regeneration of single component PLCL vascular grafts. Vascular grafts containing ECM powder (b, d) shows better cytolysis and intimal neovascularization.

### Example 2: Preparation of Polycaprolactone (PCL) and Extracellular Matrix (ECM) Composite Electrospinning Random Vascular Grafts

0.2 g ECM powder was added to the 10 ml chloroform methanol mixed solution (volume/volume=5:1), and the ECM powder was further homogenized by using a homogenizer, then 1.0 g PCL was added to the solution, stirring and dissolving overnight at room temperature to obtain a mixed solution with a concentration of 10% (mass/volume) PCL and 2% (mass/volume) ECM. The vascular grafts were prepared in the fume cupboard by electrospinning at the room temperature, and the stainless-steel receiver with 3.0 mm diameter was installed on the electrospinning machine and grounded. We inhaled the mixed solution into the syringe, installed the syringe on the injection pump, placed the syringe needle away from the receiver 15 cm, and applied 10 kV voltage to the needle using a high voltage DC power supply. The propulsion speed of the injection pump was 8 ml/h, the rotational speed of the receiver was 400 rpm, and the spinning time was 45 min. After preparation, the injection pump was removed from the electrospinning instrument and placed in a vacuum dryer to remove the spinning solution solvent. After the completion, the tubes were removed from the receiver to be the vascular grafts product.

### Example 3: Preparation of Biodegradable Polyurethane (PU) and Extracellular Matrix (ECM) Composite Casting Vascular Grafts

2.0 g ECM powder was added to the 10 ml N, N-dimethylformamide solution, and the ECM powder was further homogenized by using a homogenizer, followed by adding 0.2 g PU to the solution, stirring and dissolving at room temperature overnight to obtain a mixed solution with a concentration of PU 2% (mass/volume) and ECM 20% (mass/volume). The mixed solution was poured into a polytetrafluoroethylene (PTFE) mold with a concentric cylinder (diameter of inner cylinder was 4.0 mm and diameter of outer cylinder was 4.8 mm) and placed in a vacuum dryer to remove the solvent. After the completion, the tubes were removed from the mold to obtain the vascular grafts products.

### Example 4: Preparation of Polycaprolactone (PCL), Poly (p-Dioxanone) (PDS) and Extracellular Matrix (ECM) Composite Electrospinning Vascular Grafts

0.3 g ECM powder was added to 10 ml hexafluoroisopropanol, and the ECM powder was further homogenized by using a homogenizer. Then 1.0 g PCL and 1.0 g PDS were added to the solution, stirring and dissolving overnight at room temperature to obtain a mixed solution with 10% (mass/volume) PCL, 10% (mass/volume) PDS and 3% (mass/volume) ECM. The vascular grafts were prepared in the fume cupboard by electrospinning at the room temperature, and the stainless-steel receiver rod with 3.5 mm diameter was installed on the electrospinning machine and grounded. Inhaled the mixed solution into the syringe, installed the syringe on the injection pump, placed the syringe needle away from the receiver 10 cm, and applied 18 kV voltage to the needle using a high voltage DC power supply. The propulsion speed of the injection pump was set to 4 ml/h, the rotation speed of the receiver was 100 rpm, and the spinning time was 20 min. After the preparation was completed, it was removed from the electrospinning instrument and placed in a vacuum dryer to remove the spinning solution solvent. After the completion, the tubes were removed from the receiver to be the vascular grafts product.

### Example 5: Preparation of Electrospun Poly (1-Lactide-Caprolactone) (PLCL) and Extracellular Matrix (ECM) Composite Electrospun Vascular Grafts Made by Electrospray Polyethylene Glycol (PEO) Microspheres

0.2 g ECM powder was added to 10 ml hexafluoroisopropanol, and the ECM powder was further homogenized using a homogenizer. Then 1.5 g PLCL was added to the solution, stirring and dissolving at room temperature overnight, resulting in a mixed solution with a concentration of PLCL 15% (mass/volume) and ECM 2% (mass/volume). 20.0 g PEO was added to 10 ml trichloromethane, and the PEO was dissolved by stirring at 50° C. for 20 min. The obtained solution was cooled in an ice/water bath for 15 s until the solution became turbid. The vascular grafts were prepared in the fume cupboard by high voltage electrostatic counter spinning at the room temperature. Two kinds of liquids were inhaled into two syringes of the same specification, and the syringes were installed on two injection pumps which were axisymmetric relative to the receiver. The syringe needle equipped with PEO solution was located at the position away from the receiver 17 cm. The 17 kV voltage was applied to the needle using high voltage DC power supply, and the propulsion speed of the injection pump was set to 4 ml/h. The syringe needle with PLCL and ECM mixed solution was located at the distance from the receiver 10 cm. The high voltage DC power supply was used to apply 15 kV voltage on the needle. The injection pump propulsion speed was set to 5 ml/h, the receiver speed was 150 rpm, and the spinning time was 50 min. After the preparation was completed, the PEO microspheres were removed from the electrospinning apparatus, and then washed with 100%, 95%, 90%, 80%, 70%, and 60% gradient ethanol aqueous solutions to remove PEO microspheres out from these composites. The scaffolds were further washed with distilled water for 3 hours to completely remove PEO. The spinning solution solvent is removed in a vacuum dryer, and the vascular grafts product is obtained when the tubes were removed from the receiver.

### Example 6: Preparation of Polycaprolactone (PCL) and Extracellular Matrix (ECM) Composite Melt Spinning Vascular Grafts

1.0 g ECM powder was added to 10 ml hexafluoroisopropanol, and the ECM powder was further homogenized by using a homogenizer. Then 1.0 g PCL was added to the solution, stirring and dissolving at room temperature overnight, resulting in a mixed solution with a concentration of 10% PCL (mass/volume) and 10% ECM (mass/volume). The solvent of the mixture was removed in a vacuum dryer to obtain a composite with uniform dispersion of ECM: PCL=1:1 (mass/mass). The vascular grafts were prepared in the fume cupboard by melt spinning at the room temperature, the stainless-steel receiver with 4.0 mm diameter was installed on the melt spinning instrument, and 20.0 g ECM/PCL composite material was added to the constant temperature heating barrel. After the composite material was fully melted at 70° C., the cylinder propulsion piston speed was set to 2 ml/h, the receiver speed was set to 400 rpm, the moving speed was set to 1 mm, and the time was set to 10 min. After the completion, the tubes were removed from the receiver to be the vascular grafts product.

### Example 7: Preparation of Polycaprolactone (PCL) and Extracellular Matrix (ECM) Composite 3D Printing Vascular Grafts

1.0 g ECM powder was added to 10 ml hexafluoroisopropanol, and the ECM powder was further homogenized by using a homogenizer, and then 2.0 g PCL was added to the solution to prepare a mixed solution with a concentration of 20% PCL (mass/volume) and 10% ECM (mass/volume). The solvent of the mixture was removed in a vacuum dryer to obtain a composite with uniform dispersion of ECM: PCL=1:2 (mass/mass). The material was added to the constant temperature heating cylinder of the 3D printer, and after the material was fully melted at 70° C., the cylinder propulsion piston speed was set to 12 ml/h, and the three-dimensional moving track of the cylinder was controlled according to the pre-constructed CAD model and preset program, so as to obtain the vascular grafts with the desired three-dimensional structure. After the completion, the tubes were removed from the receiver to be the vascular grafts product.

### Example 8: Preparation of Polyglycerol Sebacate (PGS) and Extracellular Matrix (ECM) Composite Particles for Leaching Vascular Grafts

1.0 g ECM powder was added to 10 ml hexafluoroisopropanol, and the ECM powder was further homogenized using a homogenizer, then 1.0 g PGS and 0.2 g NaCl particles were added to the solution, fully mixed, stirred and dissolved overnight at room temperature to obtain a mixed solution with a concentration of 10% PGS (mass/volume) and 10% ECM (mass/volume). The mixed solution was poured into the (PTFE) mold of concentric cylinder (diameter of inner cylinder was 3.0 mm and diameter of outer cylinder was 3.7 mm) and placed in a vacuum dryer to remove the solvent. Then the scaffolds were soaked in distilled water to remove NaCl particles, and the distilled water was changed every 6 hours for 24 hours. Then the scaffolds were dried and the moisture in the scaffolds were completely removed so as to obtain the vascular grafts with the desired pore structure.

### Example 9: Preparation of Vascular Grafts Separated by Polycaprolactone (PCL), Polylactic Acid (PLA), Poly (Lactide-Glycolic Acid) Copolymer (PLGA) and Extracellular Matrix (ECM)

1.0 g ECM powder was added to 10 ml tetrahydrofuran, and the ECM powder was further homogenized using a homogenizer, then 0.5 g PLA, 0.2 g PLGA and 0.3 g PCL were added to the solution, fully mixed, stirred and dissolved overnight at 60° C. to obtain a mixed solution with concentration fractions of 5% (mass/volume) PLA, 2% (mass/volume) PLGA, 3% (mass/volume) PCL and 10% (mass/volume) ECM. The polymer blend solution was immediately cast into a preheated (60° C.) concentric cylinder (diameter of inner cylinder was 5.0 mm and diameter of outer cylinder diameter was 5.9 mm) polytetrafluoroethylene (PTFE) mold and placed in an ultra-low temperature refrigerator at −80° C. for at least 12 hours to obtain a polymer gel, then removed from the mold and immersed in the ice/water mixture to exchange tetrahydrofuran for 48 hours, and the ice/water mixture was replaced three times every 24 hours. Then the scaffolds were obtained by freeze-drying for 2 days and placed in a vacuum dryer to remove the solvent. After the completion, the tubes were removed from the mold to obtain the vascular grafts product.

Finally, it should be noted that the above embodiments are only used to illustrate the technical protocol of the invention, not to limit it; although the invention is described in detail with reference to the above-mentioned embodiments, ordinary technicians in the field should understand that they can still modify the technical protocol recorded in the above-mentioned embodiments, or replace some or all of the technical features equally. These modifications or replacements do not deviate the essence of the corresponding technical protocol from the scope of the technical protocol of the present invention embodiments.

What is claimed is:

1. A degradable synthetic polymer and natural extracellular matrix complex material involving one portion of extracellular matrix (ECM) and 0.1-10 portion of synthetic polymer by mass fraction.

2. The degradable synthetic polymers and natural extracellular matrix complex material of claim 1, wherein the synthetic polymers is consist of one or more of the following materials: polycaprolactone (PCL), poly (lactide caprolactone) copolymer (PLCL), polyurethane (PU), polyglycerol sebacate (PGS), poly (p-dioxane) (PDS), polyglycolic acid (PGA), poly (lactide)(PLA), poly (lactide glycolic acid) copolymer (PLGA), polyhydroxy fatty acid ester(PHA), polyethylene glycol(PEO).

3. The degradable synthetic polymers and natural extracellular matrix complex material of claim 1 is used for vascular grafts or vessels.

4. A preparation method of the vascular grafts or vessels of claim 3 has the following steps:
Step 1, dispose: mix the extracellular matrix and solvent based on formula quantity and make it disperse evenly then add synthetic polymers based on formula quantity and make it disperse evenly to get the mixture liquid.
Step 2, stereotype: stereotype the above mixture liquid based on a stereotyping method to get vascular grafts.

5. The preparation method for vascular grafts of claim 4, wherein the solvent is selected from one or more mixture of different ratios of tetrahydrofuran, dichloromethane, trichloromethane, acetic acid, acetone, trifluoroethanol, hexafluoroisopropanol; wherein the concentration of the extracellular matrix in Step 1 of claim 4 is 0.001-1.0 g/ml (extracellular matrix mass/solvent volume); wherein the stereotyping method in Step 2 of claim 4 includes electrospinning, wet spinning, melt spinning, 3D printing, phase separation, and particle leaching; wherein the diameter of vascular grafts obtained by the preparation methods of claim 4 for vessels is 0.5-20 mm.

6. The preparation method of the vascular grafts or vessels of claim 4, wherein when the stereotyping method is electrospinning or wet spinning, Step 2 has the following procedure: pour the mixture liquid mentioned in Step 1 into a injector, then install the injector in the microinjection pump and adjust a series of parameters including the propulsion speed of pump, the diameter, surface topography and rotation rate of receiver and its movement speed to regulate the diameter of fiber, the angle between fibers and the surface topography to get fiber tubular scaffolds which diameter of single fiber is 0.3-30 μM.

7. The preparation method of the vascular grafts or vessels of claim 4, wherein when the stereotyping method is melt spinning or 3D printing, Step 2 has the following procedure: get rid of the solvent in the mixture liquid to get polymer complex materials with uniformly dispersed ECM powder, put the complex material into a constant temperature heating barrel, heat to melt the complex material and regulate the diameter of fiber and angle between fibers by adjusting a series of parameters including three dimensional (x-axis, y-axis and z-axis) moving track of the barrel, speed of the piston, diameter of needle, rotation speed of receiver and lateral movement speed to get oriented fiber tubular scaffolds which diameter of single fiber is 10-50 μm.

8. The preparation method of vascular grafts or vessels of claim 4, wherein when the stereotyping methodis phase separation, Step 2 has the following Procedure: pour the mixture liquid mentioned in Step 1 into a special mold, control its temperature to cool and make the phase separation happen in the mixed liquid, get biphasic solid by quenching the bicontinuous polymer phase and solvent phase obtained, then remove the solvent in the solid phase by sublimating and/or solvent replacement to get porous tubular stent by controlling the quenching time and phase separation mechanism.

9. The preparation method of vascular grafts or vessels of claim 4, wherein when the stereotyping method is particle leaching method, Step 2 has the following procedure: evenly disperse pore forming agent (insoluble in mixed solution) particles with required particle size into the mixed liquid, regulate the porosity and pore size by controlling the amount and size of pore forming agent, then pour it into a special mold and removal of residual solvents from the mixture by vacuum and/or freeze drying after volatilization of the solvents to get dry polymer composites dispersed with ECM powder and pore forming agent, finally use the leaching solvents (insoluble polymer) to leach out the pore forming agent in the composite materials and dry in vacuum to get porous tubular stents.

10. The preparation method of vascular grafts or vessels of claim 9, wherein the pore forming agent is selected from sodium chloride, polyethylene glycol (PEG), maltose and glucose; wherein the leaching solvent is selected from water and/or gradient ethanol.
